(19) United States
(12) Patent Application Publication
(10) Pub. No.: US 2006/0045905 A1
(43) Pub. Date: Mar. 2, 2006

(54) Composition for repressing transformation growth factor beta

(75) Inventors: Makoto Ozeki, Yokkaichi (JP); Nobuyuki Aoi, Yokkaichi (JP)

Correspondence Address: BURR & BROWN, PO BOX 7068, SYRACUSE, NY, 13261-7068, US

(73) Assignee: Taiyokagaku Co., Ltd., Yokkaichi (JP)

(21) Appl. No.: 11/209,126

(22) Filed: Aug. 22, 2005

(30) Foreign Application Priority Data
Aug. 25, 2004 (JP) 2004-245916

(51) Int. Cl. A61K 31/522; A61K 47/00

(52) U.S. Cl. 424/439; 514/263.34

(57) ABSTRACT

A composition for repressing transformation growth factor β contains theanine as an active substance. The composition is useful for preventing or treating chronic glomerulonephritis, renal interstitial fibrosis, hepatic fibrosis, hepatic cirrhosis, idiopathic interstitial pneumonia, keloid, hidebound disease, arterial sclerosis, myocardial infarction, cardiac fibrosis, restenosis, acute megakaryoblastic leukemia, adult T-cell leukemia, chronic fatigue syndrome or ordinary fatigue.

### BACKGROUND OF THE INVENTION

1. Field of the Invention

This invention relates to a composition for repressing transformation growth factor β, containing theanine as an active substance.

2. Description of the Related Art

A transformation growth factor β (hereinafter “TGF-β”) was found as a factor promoting growth of fibroblast in 1982. A further research proved that the TGF-β was a homodimer having a molecular weight of 25 kDa and composed of two peptides each comprising 112 amino acids and bonding to each other by disulfide bond. It has now turned out that the TGF-β acts as a growth repressor for a number of cells as well as fibroblast. Typical physiological functions of the TGF-β include repressing growth of blood cells, repressing growth and function of immune cells, promoting production of extracellular matrix, repression of apoptosis inducing of Th2 cells and the like.

Furthermore, reports have been made suggesting relations between TGF-β and various diseases such as chronic glomerulonephritis, diabetic nephropathy, liver cirrhosis, idiopathic interstitial pneumonia, arterial selerosis etc. Refer to YOSHIMURA, Ashio, “Cytokine and Abnormality of Growth Factor in liver,” in Bio Clinica, 12 (6), 40, (1997); YAMAMOTO, T., NAKAMURA, T., et al., “Expression of transforming growth factor beta is elevated in human and experimental diabetic nephropathy” Proc National Academy Science U.S.A. 90 (5), 1814-8 (1993); Castilla, A., J. Prieto, et al., “Transforming growth factors beta 1 and alpha in chronic liver disease: Effects of interferon alfa therapy,” N Eng J Med 324 (14): 933-40 (1991); and Broekelmann, T. J., A. H. Limper, et al. “Transforming growth factor beta 1 is present at sites of extracellular matrix gene expression in human pulmonary fibrosis,” Proc Natl Acad Science, U.S.A., 88 (15): 6642-6.

More recently, the TGF-β is regarded as one of causative agents for chronic fatigue syndrome (hereinafter, “CFS”) which is a pathological fatigue. The CFS occurs to a person is living healthily and causes ill-defined strong whole-body dullness, slight fever, headache, lassitude, thinking power problems, neurological symptom such as blues, etc. Since such symptom continues for a long time, a healthy social life cannot be led. Atsuo KURA et al. report that blood serum TGF-β is increased in a large majority of CFS patients, and it is considered that a principal part of cascade of CFS occurrence is occupied by TGF-β. Accordingly, a method employing control of cerebrospinal fluid and TGF-β in the blood has been desired as one of treatments of CFS. Furthermore, the TGF-β is considered to be applied to general fatigue as well as to the pathological fatigue. Thus, the method of controlling TGF-β has been desired in a wider range of fields.

Hitherto found substances repressing TGF-β include high molecular protein such as anti-TGF-β immune body (refer to Yamaguchi, Y., D. M. Mann, et al., “Negative regulation of transforming growth factor-beta by the proteoglycan decorin,” Nature 346 (6281): 281-4, (1990); and JP-A-2001-206899), imide/amide ethel compound (refer to WO098/24763), phenylacetate and derivative thereof (refer to JP-A-2001-253821), and peptide (refer to WO2002/026935) comprising leucine, phenylalanine, α-aminoisobutyric acid, N-methylalanine and N-methylisoleucine.

However, anti-TGF-β immune body and peptide comprising leucine, phenylalanine, α-aminoisobutyric acid, N-methylalanine and N-methylisoleucine are digested in digestive organs. Accordingly, the effects of these substances are not expected in oral administration and a manner of administration is limited to intravenous injection, for example. Furthermore, since imide/amide ether compound, phenylacetate and derivative thereof are compounds, these compounds have problems in safety.

Thus, a composition has been desired whose safety as a TGF-β production repressor has been verified and which can be ingested by oral administration. However, no such composition has been found.

### SUMMARY OF THE INVENTION

Therefore, an object of the present invention is to provide a composition for repressing TGF-β, containing theanine as an active substance.

The inventors made every research to overcome the aforesaid problem and found that theanine which was amino acid contained in tea had an action of repressing TGF-β, completed the invention.

More specifically, the present invention is a composition for repressing TGF-β, containing theanine as an active substance.

Theanine used in the invention is a glutamic acid derivative contained in tea leaves and a principal component of deliciousness of tea. Theanine is used as a food additive for use as gustatory. Methods of producing theanine used in the invention include a method of extracting theanine from tea leaves, a method of obtaining theanine by organic synthesis reaction (Chem. Pharm. Bull., 19 (7) 1301-1307 (1971), a method of obtaining theanine by causing glutaminase to react to a mixture of glutamine and ethylamine (JP-B-H07-55154), a method of culturing cultured cells of tea in culture medium containing ethylamine and enhancing growth of cultured cells while an amount of theanine accumulated in the cells is increased (JP-A-H05-123166), a method of obtaining theanine by substituting an ethylamine derivative such as ethylamine hydrochloride for ethylamine as in JP-B-H07-55154 or JP-A-H05-123166, for example. Theanine may be produced by any one of these methods or another method. Green tea, oolong tea, black tea or the like may be exemplified as tea leaves. Any one of L-, D- and DL-theanine may be used. L-theanine is preferable in the invention since it is particularly recognized as food additives and is economic in use.

Theanine used in the invention has a high security. For example, in an acute toxicity test with use of mice, no mice died and abnormality was found in an ordinary state, weight and the like even in the case of oral administration of theanine by 5 g/kg. Furthermore, theanine is known as a principal component of deliciousness of tea and used as a food additive for use as gustatory. An amount of theanine to be added is not limited under the Food Sanitation Law. Moreover, differing from conventional medical substances, theanine has no adverse side effect. Consequently, the composition of the invention can be used as a safe and effective composition for repressing TGF-β.

As described above, there is no upper limit in an amount of theanine from the standpoint of safety. However, from the standpoint of economy, an amount of theanine to be actually ingested per time ranges from 0.1 mg/kg per weight to 100 mg/kg per weight. An amount of theanine to be ingested preferably ranges from 0.5 mg/kg per weight to 50 mg/kg per weight. An amount of theanine to be ingested more preferably ranges from 1 mg/kg per weight to 30 mg/kg per weight. Theanine used in the invention may be a refined product (containing 98% theanine or more), coarse product (containing 50% to 98% theanine), extract (containing 10% to 50% theanine) or the like.

TGF-β in the invention is a protein composed of 112 amino acids and has a molecular weight of about 25,000.

The composition of the invention has, as specific effects on the basis of TGF-β repressing activity, preventing or treatment effects against chronic glomerulonephritis, renal interstitial fibrosis, hepatic fibrosis, hepatic cirrhosis, idiopathic interstitial pneumonia, keloid, hidebound disease, arterial sclerosis, myocardial infarction, cardiac fibrosis, restenosis, acute megakaryoblastic leukemia, adult T-cell leukemia, diseases such as chronic fatigue and ordinary fatigue.

Types of foods to digest TGF-β repressing composition include theanine itself, dried food containing theanine, liquid food such as supplement, refreshing drinks, mineral water, favorite beverage and alcoholic drinks, tablets, capsule, powder, granules, health drinks and the like.

There is no specific limitation to beverages exemplified herein. However, the beverages may include tea such as green tea, oolong tea, black tea and herb tea, syrup, concentrated juice, concentrated reduced juice, straight juice, fruit juice, granule-containing fruit juice, fruit juice containing beverage, fruit-vegetable-mixed juice, vegetable juice, carbonated drink, refreshing drink, Japanese saké, beer, wine, cocktail, distilled rice spirit (or “shochu” in Japanese), whisky and so on.

Furthermore, the composition of the invention can be used with materials such as herbal medicine, herb, amino acid, vitamin, mineral and other materials allowed for use with food. There is no specific limitation to such herbal medicine. However, for example, the herbal medicine may include Valeriana fauriei, Angelicae radix, Paeoniae radix, Paeonia suffruticosa and ginseng.

There is no specific limitation to the herb. However, for example, the herb may include anise, carrot seed, cloves, coriander, cypress, cinnamon, juniper, ginger, sweet orange, pinus sylvestris, basil, patchouli, bitter orange, fennel, black pepper, bay, peppermint, bergamot, mandarin, myrrh, lemon grass, rosemary, vanilla, hyssop, eucalyptus, lime, lemon, ylangylang, cardamom, clarysage, jasmine, geranium, Bulgarian rose, rose, olibanum, matricaria, sandalwood, verbena, petit grain, vetivera zizanoides, marjoram, Melissa officinelis, rosewood, hypericum erectum, hypericum perforatum and kava kava. These herbs may be extract, essential oil, herb tea or the like as its form.

There is no specific limitation to the amino acid to be used. However, for example, the amino acid may include glutamine, glutamine acid, inosinic acid, alanine, arginine, asparaginic acid, threonine, serine, taurine, thiotaurine and hypotaurine.

The vitamin to be used may include vitamin A, vitamin B1, vitamin B2, vitamin B6, vitamin B12, vitamin C, vitamin D, vitamin E, vitamin K, folic acid, niacin, lipoic acid, pantothenic acid, biotin, ubiquinone and prostaglandin. The vitamins include the derivatives thereof. However, the vitamins to be used should not be limited to these vitamins.

The mineral may include calcium, iron, magnesium, copper, zinc, selenium and potassium. However, the mineral should not be limited to these minerals.

Furthermore, the other material allowed to be contained in food may include aloe, royal jelly, placenta, propolis, isoflavone, soy isoflavone, egg yolk lecithin, lecithin, chondroithin, cacao mass, collagen, vinegar, chlorella, spirulina, ginkgo leaf, green tea, hardy rubber tree, oolong tea, mulberry leaf, Rubus suavissimus, Lagerstroemia speciosa, unsaturated fatty acid, saccharide such as sugar alcohol and oligosaccharide, fungi such as bifidus bacillus, mushrooms such as agaricus, agaricus blazei Murrill, blacket fungus of the genus Fores, Grifola frondose, fruit such as blueberry, prune, grape, olive and plum, molokheiya such as peanut, almonde, sesame and pepper, vegetables such as green pepper, cayenne pepper, welsh onion, pumpkin, gourd, carrot, burdock, molokheiya, garlic, beefsteak plant, Japanese horseradish, tomato, scallion, leaf vegetables, sweet potato and beans, seaweeds such as “wakame” seaweed, fish and shellfish, meat of beast, birds and whales and grains. Furthermore, usable are extracts, dried products, coarse product, refined product, processed product and distilled product. However, the material should not be limited to these materials.

Medical supplies may include an internal medicine, injection medicine, pasting, suppository and inhalation medicine. However, there is no limitation to them. The internal medicine may include conventionally used tablet, capsulation, powder, granule and drink insecticide. The injection medicine may include intramuscular injection, intracutaneous injection, hypodermic injection and intravenous injection. The pasting medicine may include a mixture comprising a known carrier conventionally used for suppository and effective component of the invention and sheet to which the mixture is applied. The suppository medicine may include a mixture of the composition of the invention and conventionally used glycerogelatin, sodium stearate or propylene glycol monostearate. The inhalation medicine may include one having such a formulation as to be absorbed through nare or buccal cavity into the body with moisture or air in a conventional inhalation manner, for example.

A method of manufacturing the TGF-β repressing composition of the invention should not be limited specifically but may include ordinary manufacturing food or medicines such as a method of blending theanine and other material in the form of powder, a method of obtaining a mixed solution by dissolving theanine and other material in a solvent, a method of freezing and drying the mixed solution, a method of spray drying the mixed solution and the like.

The food in the invention may be, in a form, a solution, suspended substance, powder or solid but should not be limited to them. The food may include fish paste, processed soy beans, flavor enhancement, mousse, jelly, ices, candy, chocolate, chewing gum, cracker, cake, bread, soup, coffee, cocoa, black tea, green tea, juice, beverage produced from milk, foodstuffs produced from milk, liquor, tablet, capsule, medical supplies and the like. The invention will now be described by way of embodiment but the invention should not be limited to the embodiment.

TGF-β in the blood and cerebrospinal fluid is repressed by administration of theanine. Consequently, preventive effect and treatment effect can be achieved regarding diseases resulting from TGF-β, for example, chronic glomerulonephritis, renal interstitial fibrosis, hepatic fibrosis, hepatic cirrhosis, idiopathic interstitial pneumonia, keloid, hidebound disease, arterial sclerosis, myocardial infarction, cardiac fibrosis, restenosis, acute megakaryoblastic leukemia, adult T-cell leukemia, diseases such as chronic fatigue and ordinary fatigue.

### BRIEF DESCRIPTION OF THE DRAWINGS

Other objects, features and advantages of the present invention will become clear upon reviewing the following description of an embodiment with reference to the accompanying drawings, in which:

FIG. 1 is a graph showing TGF-β1 concentration in blood serum in each of a control group (distilled water), a theanine administration group and no exercise load group; and

FIG. 2 is a graph showing TGF-β1 concentration in cerebrospinal fluid of a control group (distilled water), a theanine administration group and no exercise load group.

### DETAILED DESCRIPTION OF THE INVENTION

Embodiments of the present invention will be described in detail. However, the technical scope of the invention should not be limited by the following description of embodiments but can be practiced in various modified forms. Furthermore, it is noted that the technical scope of the invention should encompass the scope of equivalence.

### Embodiment 1

### Manufacturing Theanine by an Enzyme Method

0.3 M glutamine and 1.5 M methylamine hydrochloride were reacted in the presence of 0.3 U glutaminase (commercially available) at 30° C. for 22 hours in a buffer solution of 0.05 M boric acid (pH 11), whereby 225 nm theanine was obtained. Reaction liquid was applied to Dowex 50×8 columnar chromatgraphy and Dowex 1×2 columnar chromatography (both made by Muromachi Chemical Co., Ltd.) thereby to be processed by ethanol, whereby an object substance is isolated from the reaction liquid.

The isolated substance was applied to an amino acid analyzer (made by Hitachi Co.) and paper chromatography. Since the isolated substance behaved in the same way as a standard substance, it was recognized as L-theanine. When the isolated substance was processed by hydrolysis using hydrochloric acid or glutaminase, glutamine acid and ethylamine were produced in a ratio of 1:1. Thus, since the isolated substance was hydrolyzed by glutaminase, it was shown that ethylamine was γ-ethylamine of glutamine acid. Furthermore, it was confirmed on the basis of glutamate dehydrogenase that glutamine acid produced by hydrolysis was L-glutamine acid. As a result, 8.5 g theanine was obtained.

### Embodiment 2

### Extraction of Theanine from Tea Leaves

10 kg tea leaf (Camellia sinensis) was extracted using heated water and thereafter, the obtained extract was passed through a cation exchange resin (type HCR W-2 made by Muromachi Chemical Industry Co., Ltd.) so as to be eluted by 1N NaOH. Eruted fraction was passed through activated charcoal (Taiko activated charcoal SG made by Futamura Chemical Industry Co., Ltd. The fraction eruted by 15% ethanol was concentrated using an RO film (type NTR 729 HF made by Nitto Denko Corporation). The concentrated eruted fraction was refined by columnar chromatography and then re-crystallized such that 24.8 g L-theanine was obtained.

L-theanine (commercial name: Suntheanine, manufactured by Taiyo Kagaku Co., Ltd.) was used in the following tests and in the manufacture of each composition.

Test Sample 1: Test on Repression of TGF-β1

Male Sprague-Dawley (SD) rats at age of 5 weeks with respective weights ranging from 220 to 270 g were purchased and fed with cubed diet (“Labo MR Stock” manufactured by Nosan Corporation) and drinking water (tap water) for a week, thereafter applied to an experiment. An aqueous solution of L-theanine was given to the rats by a sonde by 0.05, 0.1, 0.5, 1, 5, 10, 100 and 500 mg per kg of weight. Subsequently, the rats were caused to exercise on a treadmill (KN-73 treadmill manufactured by Furuya Co., Ltd.) at 5 m/min. for 3 hours. Thereafter, cerebrospinal fluid and blood were collected in an ordinary manner. As control groups were used a group of rats which were fed with distilled water instead of theanine and another group of rats which did no exercise. Furthermore, each test group consisted of five rats.

Test Sample 2: Measurement of TGF-β1 Concentration

TGFβ1 Emax™ Immuno Assay System (produced by Promega Corporation) was used for measurement of TGF-β1 concentration. Each well of a flat-bottom 96 well ELISA plate (MaxiSorp, produced by Nunc A/S) was added with a mixture of 10 μl anti-TGF-β coat mAB and 10 ml carbonate coating buffer by 100 μl. The plate was sealed by a plate sealer and incubated at 4° C. one night. After coated liquid was removed, 270 μl block 1× buffer was added and the plate was incubated at 37° C. for 35 minutes. The plate was washed with TBST wash buffer 5 times.

Blood was decomposed into blood serum, whereas precipitate was eliminated from cerebrospinal fluid by centrifugal separation. A standard solution of TGFβ1, diluted blood serum and cerebrospinal fluid were added to each well each by 100 μl. The plate was then sealed by a plate sealer and incubated at the room temperature for 1.5 hours while being shaken. After the plate was washed with TBST wash buffer 5 times, 100 μl mixture of 10 μl anti-TGF-β coat pAb and 10 μl TGFβ sample 1× buffer was added to each well and the plate was then sealed by the plate sealer. The plate was then incubated at the room temperature for 2 hours while being shaken. After the plate was washed with the TBST wash buffer 5 times, 100 μl mixture of 100 μl TGFβ HRP conjugate and 9.9 ml TGFβ sample 1× buffer was added to each well. The plate was then sealed by the plate sealer and incubated at the room temperature for 2 hours while being shaken. 100 μl TMB (room temperature) one solution was added to each well and incubated at the room temperature for 15 minutes. 100 μl 1N hydrochloric acid was added to each well so that the reaction was stopped. Within 30 minutes after reaction stop, absorbance at 450 nm was measured with a plate reader. TGF-β1 concentrations of blood serum and cerebrospinal fluid were measured from calibration curves respectively.

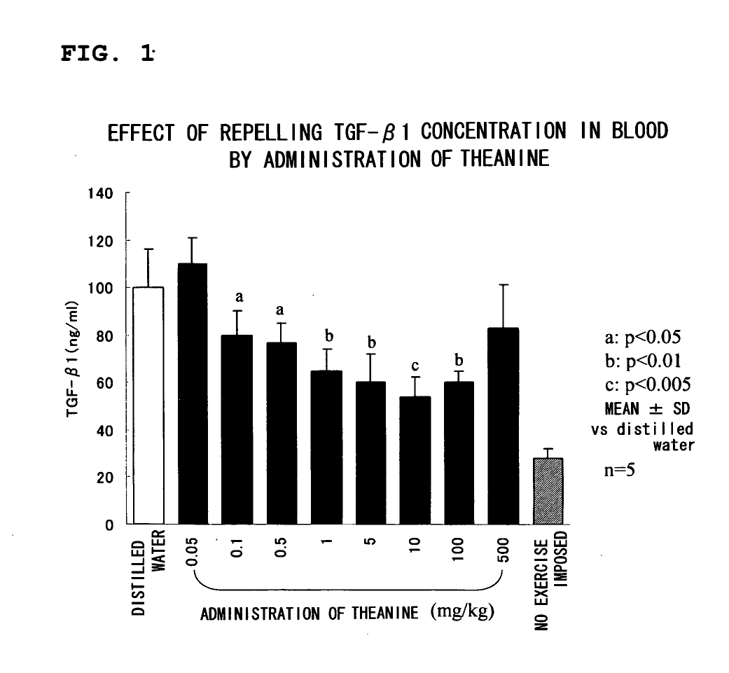

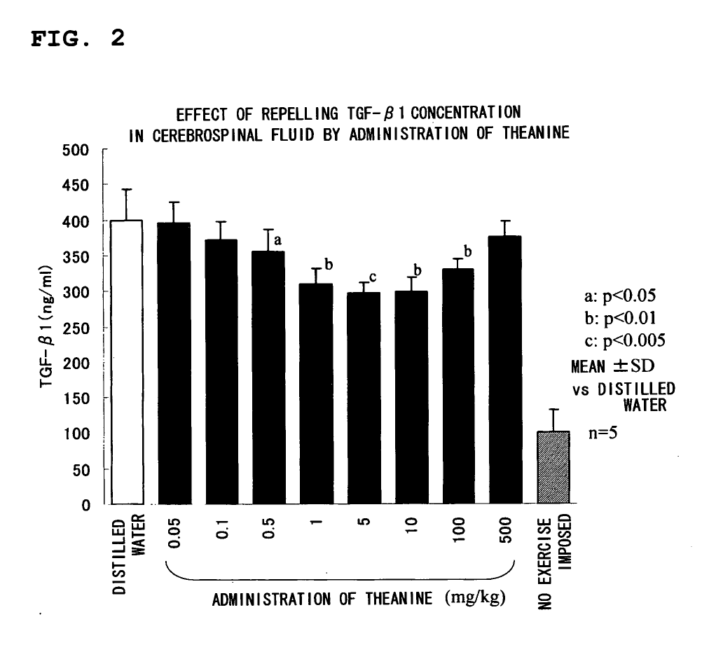

FIG. 1 shows TGF-β1 concentration of blood serum in each test group. FIG. 2 shows TGF-β1 concentration of cerebrospinal fluid in each test group. As obvious from the figures, the TGF-β1 concentrations of blood and cerebrospinal fluid are increased in the rat group which was fed with distilled water and on which exercise was imposed as compared with the rat group on which no exercise was imposed.

As obvious from FIG. 1, the TGF-β1 concentration of blood was significantly repressed in the rat group to which 0.1 to 100 mg/kg theanine was given than in the control group which was fed with distilled water. Furthermore, as obvious from FIG. 2, the TGF-β1 concentration of cerebrospinal fluid was significantly repressed in the rat group to which 0.5 to 100 mg/kg theanine was given than in the control group which was fed with distilled water.

The foregoing test examples state that administration of theanine has an effect of repressing the TGF-β1 concentrations of blood and cerebrospinal fluid.

### Embodiment 3

### Manufacture of Tablets Blended with Theanine

As an example of drug containing TGF-β repelling composition blended with theanine, the following materials were mixed together and thereafter made into tablets, whereby tablets blended with theanine were manufactured.

TABLE 1

| Material | Weight % | Weight (mg) |
|---|---|---|
| Frost sugar | 71.67 | 537.5 |
| Trehalose | 10.00 | 75.0 |
| L-theanine | 13.33 | 100.0 |
| Sucrose esters of fatty acids | 1.00 | 7.5 |
| Flavor (lemon flavor) | 4.00 | 30.0 |
| Total | 100.00 | 750.0 |

More specifically, the materials were mixed according to the above formulation and after granulating, were made into tablets each of which weighed 0.75 g.

### Embodiment 4

### Manufacture of Candy Blended with Theanine

As an example of food containing TGF-β repelling composition blended with theanine, candy blended with theanine was manufactured using the following materials.

TABLE 2

| Material | Weight (kg) |
|---|---|
| Granulated sugar | 64.0 |
| Starch syrup | 23.0 |
| L-theanine | 10.0 |
| Flavor (lemon flavor) | 0.05 |
| 50% tartaric acid | 1.0 |
| Water | 30.0 |

Granulated sugar was heated to 110° C. while being dissolved into 20 kg water. L-theanine dissolved by 10 kg water and starch syrup were added to heated granular sugar and the temperature was increased to 145° C. Heating was then stopped and 50% tartaric acid was added to the mixture. The mixture was cooled to the temperature from 75° C. to 80° C. and then shaped by a shaping roller, whereby candy blended with theanine was prepared.

A measurement of an amount of L-theanine contained in the candy was 89.6 mg/g per candy (1.2 g).

### Embodiment 5

### Manufacture of Blueberry Beverage Blended with Theanine

As an example of food containing TGF-β repelling composition blended with theanine, a beverage blended with theanine was manufactured using the following materials.

TABLE 3

| Material | Weight (kg) |
|---|---|
| Fructose-glucose | 12.0 |
| Concentrated blueberry juice | 1.0 |
| One-fifth transparent lemon juice | 0.4 |
| Sodium citrate | 0.05 |
| 50% sodium citrate (crystal) | for pH adjustment |
| L-theanine | 0.1 |
| Flavor (blueberry flavor) | 0.05 |
| Water | a proper amount |
| Total amount | 100.0 |

Water was added to fructose-glucose, concentrated blueberry juice, one-fifth transparent lemon juice, sodium citrate and L-theanine and dissolved through agitation. The mixture was prepared to pH 3.1 using 50% sodium citrate (crystal) and the temperature thereof was increased to 95° C. Thereafter, flavor was added to the mixture so that a total amount was 100 ml. The mixture was then cooled, whereby a blueberry beverage blended with L-theanine was manufactured. A measurement of an amount of L-theanine contained in the blueberry juice was 98.3 mg/100 ml.

### Embodiment 6

### Manufacture of Grapefruit Beverage Blended with Theanine

As an example of food containing TGF-β repelling composition blended with theanine, a beverage blended with theanine was manufactured using the following materials.

TABLE 4

| Material | Weight (kg) |
|---|---|
| Fructose-glucose | 6.0 |
| L-theanine | 0.1 |
| Ferric pyrophosphate | 0.06 |
| Placenta extract | 0.01 |
| 100% concentrated grapefruit juice | 0.1 |
| Sodium citrate | for pH adjustment |
| Flavor (grapefruit flavor) | 0.05 |
| Water | a proper amount |
| Total | 100.0 |

Fructose-glucose, L-theanine, ferric pyrophosphate, placenta extract and 100% concentrated grapefruit juice were added to water and dissolved through agitation. The mixture was prepared to pH 3.1 using sodium citrate and the temperature thereof was increased to 95° C. Thereafter, flavor was added to the mixture so that a total amount was 100 ml. The mixture was then cooled, whereby a grapefruit beverage blended with L-theanine was manufactured. A measurement of an amount of L-theanine contained in the grapefruit juice was 96.4 mg/100 ml.

Food or beverage containing no theanine was manufactured with respect to each of the foregoing embodiments 3 to 6 and compared with each of the embodiments 3 to 6 concerning the feeling of appetite and taste. As a result, no difference was found.

As obvious from the foregoing, the transformation growth factor β (TGF-β) in blood and cerebrospinal fluid is repelled by administration of theanine. Consequently, the composition of the invention has, as specific effects on the basis of TGF-β repressing activity, preventing or treatment effects against chronic glomerulonephritis, renal interstitial fibrosis, hepatic fibrosis, hepatic cirrhosis, idiopathic interstitial pneumonia, keloid, hidebound disease, arterial sclerosis, myocardial infarction, cardiac fibrosis, restenosis, acute megakaryoblastic leukemia, adult T-cell leukemia, diseases such as chronic fatigue and ordinary fatigue.

The foregoing description and drawings are merely illustrative of the principles of the present invention and are not to be construed in a limiting sense. Various changes and modifications will become apparent to those of ordinary skill in the art. All such changes and modifications are seen to fall within the scope of the invention as defined by the appended claims.

1. A composition for repressing transformation growth factor β, containing theanine as an active substance.

2. The composition according to claim 1, wherein an objective disease includes at least one of chronic glomerulonephritis, renal interstitial fibrosis, hepatic fibrosis, hepatic cirrhosis, idiopathic interstitial pneumonia, keloid, hidebound disease, arterial sclerosis, myocardial infarction, cardiac fibrosis, restenosis, acute megakaryoblastic leukemia, adult T-cell leukemia, chronic fatigue syndrome or ordinary fatigue.

3. A food containing the composition as described in claim 1.

4. A drug containing the composition as defined by claim 1.

5. A food containing the composition as described in claim 2.

6. A drug containing the composition as defined by claim 2.
